(19) United States
(12) Patent Application Publication
(10) Pub. No.: US 2024/0325479 A1
(43) Pub. Date: Oct. 3, 2024

(54) Total extract of Fructus Cnidii and its application and map construction method

(71) Applicant: Liping YANG, Guangzhou (CN)

(72) Inventor: Liping YANG, Guangzhou (CN)

(21) Appl. No.: 18/741,725

(22) Filed: Jun. 12, 2024

(30) Foreign Application Priority Data
Oct. 13, 2022 (CN) 202211256370.2

(51) Int. Cl. A61K 36/234; A61K 36/752; G01N 30/02; G01N 30/72; G01N 30/86; G01N 33/15

(52) U.S. Cl. CPC A61K 36/234; A61K 36/752; G01N 30/7233; G01N 30/8631; G01N 33/15; G01N 2030/027

(57) ABSTRACT

The invention discloses a total extract of Fructus Cnidii, which contains bergamot, the application of the total extract of Fructus Cnidii in preparing medicines for preventing and/or treating allergic dermatosis, psoriasis or herpes zoster, and the construction method of the fingerprint of the total extract of Fructus Cnidii. The invention adopts LC-MS technology to establish the fingerprint of the total extract of Fructus Cnidii, thus effectively improving the quality stability, uniformity and controllability of the total extract of Fructus Cnidii.

### TECHNICAL FIELD

The invention belongs to the technical field of Fructus Cnidii, and particularly relates to a total extract of Fructus Cnidii and its application and map construction method.

### BACKGROUND OF THE INVENTION

Traditional Chinese medicine refers to medicinal substances under the guidance of the theory of traditional Chinese medicine, which has practical and innovative material sources. The production of traditional Chinese medicine involves many links: Chinese herbal medicine resources, Chinese herbal medicine pieces (including processing), Chinese herbal medicine prescriptions, and Chinese traditional medicine preparations (Chinese patent medicines). If any link is not well controlled, it will affect the quality of products and the safety and effectiveness of clinical efficacy. Therefore, the quality control of traditional Chinese medicine is one of the key and difficult problems in the development of modern Chinese medicine. At present, in the existing standards in China, the quality standards of Chinese herbal medicines and Chinese patent medicines are mostly limited to identifying appearance, checking properties and moisture, and less effective components are detected. Even if the component determination standards are established, most of the measured components are only 1-2 representative index components, and the effective components directly related to curative effect are pain points. Therefore, how to scientifically, reasonably and comprehensively formulate the quality control standards of traditional Chinese medicine is of great significance to the quality, safety and effectiveness of traditional Chinese medicine and public health.

The fingerprint of traditional Chinese medicine is a chromatogram or spectrogram that can indicate the chemical characteristics of traditional Chinese medicine or traditional Chinese medicine preparation after proper treatment and certain analytical means. Fingerprint of traditional Chinese medicine is a comprehensive and quantifiable identification method, which is based on the systematic study of chemical components of traditional Chinese medicine and is mainly used to evaluate the authenticity, Excellence and stability of the quality of traditional Chinese medicine and its preparations. “Integrity” and “fuzziness” are its remarkable characteristics.

The fingerprint technology of traditional Chinese medicine has involved many methods, including thin layer scanning (TLCS), high performance liquid chromatography (HPLC), gas chromatography (GC) and high performance capillary electrophoresis (HPCE), and ultraviolet spectroscopy (UV), infrared spectroscopy (IR), mass spectrometry (MS), nuclear magnetic resonance (NMR) and X-ray diffraction. Among them, chromatography is the mainstream method, especially HPLC, TLCS and GC have become recognized as three conventional analytical methods. Because HPLC has the characteristics of high separation efficiency, high selectivity, high detection sensitivity, fast analysis speed and wide application range. Most components of traditional Chinese medicine can be analyzed and detected by HPLC, and rich application experience has been accumulated. Therefore, high performance liquid chromatography (HPLC) has become the first choice of fingerprint technology of traditional Chinese medicine. With the application of HPLC-MS and GC-MS, the fingerprint technology of traditional Chinese medicine is becoming more and more perfect.

At present, the effective components of the total extract of Fructus Cnidii include Zanthoxylol, Xanthotoxin, Isoanisidin, Bergamot lactone, Imperatorin, Osthole and so on. But there are still some unknown components that have not been discovered. With the progress of extraction technology and the development of detection technology, more and more effective components are being gradually extracted and further utilized.

### DESCRIPTION OF THE INVENTION

The first purpose of the present invention is a total extract of Fructus Cnidii, which contains bergamot.

The purpose of the present invention is also to provide the application of the total extract of Fructus Cnidii in preparing drug for preventing and/or treating allergic dermatosis, psoriasis or herpes zoster.

The last purpose of the present invention is to provide a method for constructing the fingerprint of the total extract of Fructus Cnidii.

The first purpose of the present invention can be achieved by the following technical scheme: a total extract of Fructus Cnidii contains bergamot.

In the present invention, a new ingredient, bergamot, is found in the traditional Chinese medicine of Fructus Cnidii, the total extract of Fructus Cnidii and the preparation of Fructus Cnidii for the first time through the construction method of fingerprint.

The total extract of Fructus Cnidii can be a commercially available extract of Fructus Cnidii, or it can be prepared by the following methods: taking the traditional Chinese medicine of Fructus Cnidii, adding ethanol with a volume percentage content of 80-85%, 3-6 times the total mass of Fructus Cnidii, and reflux extraction for 1-3 times, each time for 2-3 hours. Filter, combine the filtrate, and concentrate under reduced pressure to obtain an alcohol extract. Then, crystallize and dry to obtain the total extract of Fructus Cnidii, which is the total coumarin of Fructus Cnidii. Even better, the total extract of Fructus Cnidii is prepared by the following method: taking an appropriate amount of Fructus Cnidii medicinal material, adding 3-6 times the amount (mass) of 85% (volume percentage content) ethanol, heating and refluxing at 75° C. for 3 times, each time for 2 hours, filtering, merging the filtrate, and concentrating under reduced pressure to obtain an alcohol extract. Then, crystallization and vacuum drying are carried out to obtain the total extract of Fructus Cnidii, which is the total coumarin of Fructus Cnidii.

Further, the total extract of Fructus Cnidii also contains Zanthoxylol, Xanthotoxin, Isoanisidin, Bergamot lactone, Imperatorin, Osthole, Hesperidone hydrate, and Hesperidone.

In addition, the total extract of Fructus Cnidii in this application mainly contains total coumarin of Fructus Cnidii.

The second purpose of the present invention can be achieved by the following technical scheme: the application of the total extract of Fructus Cnidii in preparing medicines for preventing and/or treating allergic dermatosis, psoriasis or herpes zoster.

The second prupose of the present invention can be achieved by the following technical scheme: the application of the total extract of Fructus Cnidii in preparing medicines for preventing and/or treating allergic dermatosis, psoriasis or herpes zoster.

Preferably, the drug comprises a total extract of Fructus Cnidii combined with a pharmaceutically acceptable carrier or excipient.

As a preferred embodiment of the present invention, the medicine is Fructus Cnidii ointment, Fructus Cnidii spray or Fructus Cnidii liquid containing the total extract of Fructus Cnidii.

That is to say, the drugs containing total extract of Fructus Cnidii in this application are mainly external preparations of total coumarin of Fructus Cnidii, including ointment of total coumarin of Fructus Cnidii, spray of total coumarin of Fructus Cnidii, liquid of total coumarin of Fructus Cnidii, etc., which are all prepared by conventional methods in this field.

Preferably, the ointment of total coumarin of Fructus Cnidii is an external ointment made of Fructus Cnidii total coumarin and conventional pharmaceutical excipients, and the content of total coumarin in the ointment of total coumarin of Fructus Cnidii is 10% by mass.

According to the clinical trials of this traditional Chinese medicine, the preparation is preferably ointment according to its applicable objects and indications.

In order to realize the above dosage form, it is necessary to add appropriate pharmaceutical excipients, such as solubilizer, matrix, humectant, etc., when preparing the dosage form. Solubilizer includes polysorbate 80, matrix includes white vaseline, stearic acid, glycerol monostearate, and humectant includes glycerol.

More preferably, the ointment of total coumarin of Fructus Cnidii is prepared by the following methods: emulsifying and encapsulating the total coumarin raw material of Fructus Cnidii, an oil phase made of stearic acid, glycerol monostearate and vaseline, and a water phase made of glycerol, water and polysorbate 80.

Preferably, the spray of total coumarin of Fructus Cnidii is an externally applied spray prepared by ultrasonic dissolution, standing, filtering and constant volume of spray of total coumarin of Fructus Cnidii, and the mass percentage of total coumarin in the spray of total coumarin of Fructus Cnidii is 1-10%.

Preferably, the liquid of total coumarin of Fructus Cnidii is an externally applied liquid prepared from total coumarin of Fructus Cnidii and impure ethanol solvent, wherein the impure ethanol solvent includes benzyl alcohol, propylene glycol and polysorbate 80, and the mass percentage of the total coumarin in the liquid of total coumarin of Fructus Cnidii is 0.5%-5%.

The third purpose of the present invention can be achieved through the following technical solution: a method for constructing a fingerprint of the total extract of Fructus Cnidii, comprising the following steps:

(1) preparation of mixed reference solution: p-coumaric acid, osthole, isoanisidine, zanthoxyphenol, xanthotoxin, bergamot lactone and imperatorin are taken and dissolved with ethanol to obtain a mixed reference solution;

(2) Preparation of test solution: take the total extract of Fructus Cnidii, add ethanol, after ultrasonic treatment, make up the weight loss with ethanol, mix well, filter, and take the filtrate to obtain the test solution;

(3) Take the mixed reference solution and test solution, inject the sample, adopt full scanning analysis of high resolution mass spectrometry of liquid chromatography, use software to identify the common peaks of the characteristic spectrum of the test sample, extract and confirm the characteristic components, analyze the structure, identify and confirm the unique chemical components of Fructus Cnidii, obtain the chemical components of Fructus Cnidii, and establish the standard fingerprint of the total extract of Fructus Cnidii.

In the method for constructing the fingerprint of Fructus Cnidii extract:

Preferably, the preparation of the mixed reference solution in step (1) includes: accurately weighing about 1 mg of p-coumaric acid, osthole, isoanisidine, zanthoxyphenol, xanthotoxin, bergamot lactone and imperatorin, respectively, placing them in 10 mL volumetric flasks, adding ethanol to dissolve and dilute them to scale, shaking them evenly, and then accurately weighing 1.0 mL of the above solutions respectively and placing them in 10 mL.

Preferably, the volume percentage of ethanol in step (2) is 70%, the relationship between the total extract of Fructus Cnidii and ethanol is 0.05g:50 mL, and the ultrasonic treatment time is 60 min.

Preferably, the parameters of liquid chromatography in step (3) are as follows: chromatographic conditions: C18 column; Flow rate: 0.5ml/min; Column temperature: 40° C.; Wavelength: 310 nm;; Sample volume: 10 μl; Mobile phase A: 0.1% (volume percentage) acetic acid aqueous solution; Mobile phase B: methanol, gradient elution conditions are as follows:

| Time | Mobile phase | Mobile phase B |
|---|---|---|
| 0 | 95 | five |
| 10 | 65 | 35 |
| 20 | 55 | 45 |
| 35 | 55 | 45 |
| 40 | 53 | 47 |
| 65 | 53 | 47 |
| 70 | 30 | 70 |
| 75 | 10 | 90 |
| 85 | 10 | 90 |
| 85.1 | 0 | 100 |
| 120 | 0 | 100 |
| 120.1 | 95 | five |
| 130 | 95 | five |

Preferably, the chromatographic column in step (3) is Thermoscientific Acclaim TM 120C18 250× 4.6 mm, 5 μm.

Preferably, the standard fingerprint of the total extract of Fructus Cnidii in step (3) includes the following compounds: xanthotoxin, xanthotoxin, isoanisidin, bergamot lactone, imperatorin, osthole, hesperidin hydrate, hesperidone and bergamot.

This application further establishes a method for constructing fingerprint spectra of traditional Chinese medicine, total extract of Fructus Cnidii, and Fructus Cnidii preparations. The method adopts LC-MS technology to establish fingerprint spectra of traditional Chinese medicine “Fructus Cnidii”, its total extract “Fructus Cnidii coumarin” and its preparations such as “Ointment of total coumarin of Fructus Cnidii”, effectively improving the quality stability, consistency, and controllability of Fructus Cnidii, total extract of Fructus Cnidii, and their preparation products.

Ten common peaks in Fructus Cnidii can be determined by the fingerprint determination method in this application, and the compounds of seven peaks are confirmed to be p-coumaric acid, Zanthoxylol, Xanthotoxin, Isoanisidin, Bergamot lactone, Imperatorin and Osthole, respectively; Nine common peaks in Fructus Cnidii extract and Fructus Cnidii preparation can be determined, and it is confirmed that the compounds with seven peaks are Zanthoxylol, Xanthotoxin, Isoanisidin, Bergamot lactone, Imperatorin and Osthole respectively.

Specifically, the standard fingerprint of Fructus Cnidii includes the following compounds: p-coumaric acid, Zanthoxylol, Xanthotoxin, Isoanisidin, Bergamot lactone, Imperatorin, Osthole, Hesperidine hydrate, Hesperidone and Bergamot.

Specifically, the standard fingerprints of the total extract of Fructus Cnidii and the preparation of Fructus Cnidii include the following compounds: Zanthoxylol, Xanthotoxin, Isoanisidin, Bergamot lactone, Imperatorin, Osthole, Hesperidine hydrate, Hesperidone and Bergamot.

The invention has the following advantages:

(1) A new ingredient bergamot was obtained from the total extract of Fructus Cnidii;

(2) This application adopts LC-MS technology to establish a fingerprint of the total extract of Fructus Cnidii. Furthermore, the fingerprint of traditional Chinese medicine Fructus Cnidii and preparations containing Fructus Cnidii total extract, such as Ointment of total coumarin of Fructus Cnidii, can be further established based on this method, effectively improving the quality stability, uniformity, and controllability of Fructus Cnidii, Fructus Cnidii total extract, and their preparation products;

(3) In this application, the content of each component in the total extract of Fructus Cnidii was detected through the optimization of chromatographic conditions and the establishment of detection methods, and the common peaks of characteristic spectra were confirmed by modern scientific identification means (LC-MS, etc.), the attribution of each component was confirmed, the fingerprint of the total extract of Fructus Cnidii was established, and the fingerprint of Chinese medicinal materials and the preparation of the total extract of Fructus Cnidii was further established, thus improving the controllability of product quality.

### BRIEF DESCRIPTION OF DRAWINGS

FIG. 1 is the fingerprint of the mixed reference substance in Example 3;

FIG. 2 is the fingerprint of the reference medicinal materials in Example 3, in which the compounds represented by the peak numbers are peak 1 p-coumaric acid, peak 2 Zanthoxylol, peak 3 hesperidin hydrate, peak 4 Xanthotoxin, peak 5 isoanisidine, peak 6 Hesperidone, peak 7 bergamot lactone, peak 8 imperatorin, peak 9 osthole and peak 10 bergamot respectively;

FIG. 3 is the fingerprint of Fructus Cnidii in Example 3, and the peak number 1-10 is the same as FIG. 2;

FIG. 4 is the fingerprint of the total extract of Fructus Cnidii (namely, total coumarin of Fructus Cnidii) in Example 3, with peak 1 Zanthoxylol, peak 2 hesperidin hydrate, peak 3 Xanthotoxin, peak 4 isoanisidine, peak 5 Hesperidone, peak 6 bergamot lactone, peak 7 imperatorin, peak 8 osthole and peak 9 bergamot.

FIG. 5 is the fingerprint of the ointment of total coumarin of Fructus Cnidii in Example 3, and the peak number 1-9 is the same as FIG. 4;

FIG. 6 is a comparative chromatogram of the fingerprints of 15 batches of Chinese medicinal materials in Example 3;

FIG. 7 is the fingerprint contrast chromatogram of 15 batches of Chinese

herbal medicine extracts in Example 3;

FIG. 8 is a comparative chromatogram of 15 batches of samples of the preparation made of Chinese herbal medicine extracts in Example 3;

FIG. 9 is the mass spectrum of unknown peak No. 10 of Fructus Cnidii in Example 3;

FIG. 10 is an ion current diagram of the unknown peak No. 10 of Fructus Cnidii in Example 3.

FIG. 11 is the mass spectrum of unknown peak No. 10 of Fructus Cnidii in Example 3;

FIG. 12 is the secondary mass spectrum of unknown peak No. 10 of Fructus Cnidii in Example 3;

FIG. 13 is the standard mass spectrum of bergamot in Example 3;

FIG. 14 is a structural diagram of bergamot in Example 3;

FIG. 15 is a picture of the effect of the ointment of total coumarin of Fructus Cnidii on herpes zoster in Example 4;

FIG. 16 is a picture of the effect of the ointment of total coumarin of Fructus Cnidii on abdominal psoriasis in Example 4;

FIG. 17 is a picture of the effect of the ointment of total coumarin of Fructus Cnidii on back psoriasis in Example 4;

FIG. 18 is a picture of the effect of the ointment of total coumarin of Fructus Cnidii on atopic dermatitis of knee socket in Example 4;

FIG. 19 is a picture of the effect of the ointment of total coumarin of Fructus Cnidii on ear atopic dermatitis in Example 4.

### DETAILED DESCRIPTION OF EMBODIMENTS OF THE INVENTION

The present invention will be further described with specific examples, so that those skilled in the art can better understand the present invention, but it is not limited by this. The raw materials used below are all from commercial channels unless otherwise specified.

### Example 1 Preparation of Fructus Cnidii Extract

Preparation method: Take 1 kg of the traditional Chinese medicine Fructus Cnidii and grind it into coarse powder. Place it in an extractor, add 6 times the mass of 85% (volume percentage content) ethanol solvent, heat and reflux for 2 hours, collect the extraction solution, and then add 3 times the mass of 85% (volume percentage content) ethanol solvent to extract twice using the same method. Combine the extraction solution, leave it overnight at room temperature, and concentrate it under reduced pressure at 80° C. to obtain the extract. After 24 hours at room temperature, precipitate a green precipitate, filter it out, and dry it to obtain the total extract of Fructus Cnidii, which is the total coumarin of Fructus Cnidii.

### Example 2 Preparation of Ointment

Preparation method: The total coumarin of Fructus Cnidii obtained by the preparation method of Example 1 will be combined with a suitable matrix to form a paste like external ointment, which is an oil in water formulation. The formula is: 100 g of total coumarin of Fructus Cnidii, 80 g of stearic acid, 100 g of monostearic acid glyceride, 80 g of white vaseline, 160 g of glyceride, 60 g of polysorbide-80, 280 g of water, glycerol: water (4:10) added to 1000 g to obtain the Fructus Cnidii ointment formulation.

Experimental example: The following methodological studies were conducted using traditional Chinese medicine (Fructus Cnidii), total extract samples of Fructus Cnidii prepared in Example 1, and Fructus Cnidii ointment preparation samples prepared in Example 2. Verify the method in terms of system applicability, specificity, repeatability, intermediate precision, stability, etc.

### Example 3

### 1. Chromatographic Conditions

1.1 Instruments: Thermo Fisher Scientific Vanquish HPLC, UV detector;

1.2 chromatographic conditions: chromatographic column Agilent ZORBAX Eclipse plus C18, 4.6*250 mm, 5um: No.: GB-L-20-02-192;

The following parameters were set as initial conditions, and the flow rate was 0.5 mL/min; Column temperature: 40° C.; The wavelength is 310nm; Mobile phase A: 0.1% acetic acid aqueous solution; Mobile phase B: methanol; Gradient elution. The elution procedure is shown in Table 1 below:

TABLE 1
mobile phase gradient elution procedure

| Time (min) | Mobile phase A (%) | Mobile phase B (%) |
|---|---|---|
| 0 | 95 | 5 |
| 10 | 65 | 35 |
| 20 | 55 | 45 |
| 35 | 55 | 45 |
| 40 | 45 | 55 |
| 50 | 45 | 55 |
| 55 | 15 | 85 |
| 60 | 10 | 90 |
| 65 | 10 | 90 |
| 65.1 | 95 | 5 |
| 70 | 95 | 5 |

### 2. Optimization of Chromatographic Conditions

### 2.1 Optimization of Chromatographic Conditions

Screening the dominant components (osthole, zanthoxylol, xanthotoxin, bergamot lactone, imperatorin, isoanisidin, p-coumaric acid) that can be identified by the reference substance with high content in Fructus Cnidii samples. First, using the basic chromatographic conditions (Table 1), and taking the sum of their peak areas as the index, and at the same time investigating the separation degree among the components, the conditions such as chromatographic column screening and flow matching ratio screening were carried out. Finally, the aim is to make the dominant components p-coumaric acid, zanthoxylol, xanthotoxin, bergamot lactone, imperatorin, isoanisidin and osthole well separated from each other, and the above dominant components can be well separated from the adjacent components in the sample. See Table 2 for the optimized chromatographic conditions.

TABLE 2
Optimized chromatographic conditions

| Chromatographic | Thermo Scientific Acclaim ™ 120 | | |
|---|---|---|---|
| column | C18 250 × 4.6 mm, 5 μm; No.: | | |
| | GB-L-20-02-036 | | |
| Current velocity | 0.5 mL/min | | |
| Column temperature | 40° C. | | |
| Injection volume | 10 μL | | |
| Detection wavelength | 310 nm | | |
| Mobile phase A | 0.1% acetic acid | | |
| Mobile phase B | methanol | | |
| Mobile phase | Time | Mobile phase | Mobile phase |
| gradient | (min) | A (%) | B (%) |
| | 0 | 95 | 5 |
| | 10 | 65 | 35 |
| | 20 | 55 | 45 |
| | 35 | 55 | 45 |
| | 40 | 53 | 47 |
| | 65 | 53 | 47 |
| | 70 | 30 | 70 |
| | 75 | 10 | 90 |
| | 85 | 10 | 90 |
| | 85.1 | 0 | 100 |
| | 120 | 0 | 100 |
| | 120.1 | 95 | 5 |
| | 130 | 95 | 5 |

### 3. Experiment of Extraction Conditions

3.1 Sample treatment method: Take about 0.5 g of Fructus Cnidii, about 50 mg of Fructus Cnidii total coumarin raw material and about 0.1 g of ointment of total coumarin of Fructus Cnidii respectively, and optimize the extraction method through orthogonal test, including the investigation of extraction solvent, extraction solvent concentration, extraction method, extraction time and extraction solvent volume. See Table 3 for the conditions.

TABLE 3
Experiment of Optimizing Extraction Conditions

| | Solvent | | | Solvent volume |
|---|---|---|---|---|
| No. | extraction | Time (min) | Extraction method | (mL) |
| 1 | Water | 10, 20, 30, 45, 60 | reflux method, ultrasonic method | 10, 20, 50, 100 |
| 2 | 99.9% methanol | 10, 20, 30, 45, 60 | reflux method, ultrasonic method | 10, 20, 50, 100 |
| 3 | 70% methanol | 10, 20, 30, 45, 60 | reflux method, ultrasonic method | 10, 20, 50, 100 |
| 4 | 99.99% ethanol | 10, 20, 30, 45, 60 | reflux method, ultrasonic method | 10, 20, 50, 100 |
| 5 | 70% ethanol | 10, 20, 30, 45, 60 | reflux method, ultrasonic method | 10, 20, 50, 100 |

### 3.2 Test Conclusions

(1) When 70% (volume percentage) ethanol is used as the extraction solvent of medicinal materials, its “total peak area/sample weight” value is the largest, that is, the extraction efficiency is the highest; When methanol, 70% methanol, ethanol and 70% ethanol are used as extraction solvents for raw materials and preparations, the “total peak area/sample weight” is basically the same. Considering the toxicity and economy of solvents, 70% ethanol is selected as extraction solvent.

(2) The total peak area/sample weight of ultrasonic extraction of medicinal materials and preparations is slightly higher than that of heating reflux extraction, and the total peak area/sample weight of raw materials is slightly lower than that of heating reflux extraction. There is no obvious difference in extraction ability between the two extraction methods. Considering the simplicity of operation, ultrasonic treatment is selected as the extraction method.

(3) The value of “total peak area/sample weight” of medicinal materials is the largest after ultrasonic treatment for 60 min, that is, the extraction efficiency is the highest; The total peak area/sample weight of API in 10˜60 min is basically the same; The total peak area/sample weight of the preparation in 20˜60 min is basically the same. Combined with the experimental results of medicinal materials, raw materials and preparations, the final extraction time was ultrasonic 60 min.

(4) With the increase of extraction solvent, the “total peak area/sample weight” also increases when the concentration is consistent. Considering the response of each characteristic peak, the volume of extraction solvent is finally selected to be 50 mL.

### 4. Preparation of Test Sample

Through the experiment of extraction conditions, the preparation method of the test solution was determined as follows:

The preparation method of the sample of characteristic spectrum of Fructus Cnidii is as follows: take about 0.5 g of coarse powder of Fructus Cnidii, weigh it accurately, add 50 mL of 70% ethanol, weigh it, after ultrasonic treatment for 60 min, weigh it again after the solution is cooled, make up the lost weight with 70% ethanol, shake it evenly, filter it with microporous membrane, and take the filtrate.

The preparation method of the sample of characteristic spectrum of total coumarin in Fructus Cnidii is as follows: take about 50 mg of total coumarin in Fructus Cnidii (that is, total extract of Fructus Cnidii), weigh it accurately, add 50 mL of 70% ethanol, weigh it, after ultrasonic treatment for 60 min, weigh it again after the solution is cooled, make up the lost weight with 70% ethanol, shake it evenly, filter it with microporous membrane, and take the filtrate.

The preparation method of the test sample of total coumarin ointment of Fructus Cnidii is as follows: take about 0.1g of total coumarin ointment of Fructus Cnidii, accurately weigh it, accurately add 50 mL of 70% ethanol, weigh it, after ultrasonic treatment for 60 min, weigh it again after cooling the solution, make up the lost weight with 70% ethanol, shake it evenly, filter it with microporous membrane, and take the filtrate.

### 4.1 Reference Substance and Sample Source Information

### 4.1.1 Reference Substance

Zanthoxylol bungeanum was purchased from Dulemeitian Pharmaceutical Technology Co., Ltd., p-coumaric acid, xanthotoxin, isoanisidin, bergamot lactone and imperatorin were purchased from Chengdu Glip Biotechnology Co., Ltd., and osthole and Fructus Cnidii were purchased from China Institute for Drug and Biological Products Inspection (Central Inspection Institute).

### 4.1.2 Sample

p-coumaric acid (99. 97%, purity, the same below), xanthotoxin (99. 88%), isoanisidin (99. 97%), bergamot lactone (99. 85%) and imperatorin (98. 24%) were purchased from Dulemeitian Pharmaceutical Technology Co., Ltd.

### 4.1.2 Sample

Methods The samples of Fructus Cnidii, raw materials of total coumarin of Fructus Cnidii and total coumarin of Fructus Cnidii of ointment were from Guangdong HiST Pharmaceutical Co., Ltd.

Samples for methodology verification and samples for fingerprint determination of Fructus Cnidii were purchased from Anhui and Guangzhou, and raw materials of Fructus Cnidii total coumarin, ointment of total coumarin of Fructus Cnidii and blank ointment of total coumarin of Fructus Cnidii were purchased from Guangdong HiST Pharmaceutical Co., Ltd.

The raw materials of total coumarin from Fructus Cnidii and Ointment of total coumarin of Fructus Cnidii can also be prepared according to the methods in Examples 1-2.

### 4.2 Preparation of Reference Substance and Sample

### 4.2.1 Preparation of Mixed Reference Substance

Accurately weigh about 1 mg of p-coumaric acid, osthole, isoanisidine,

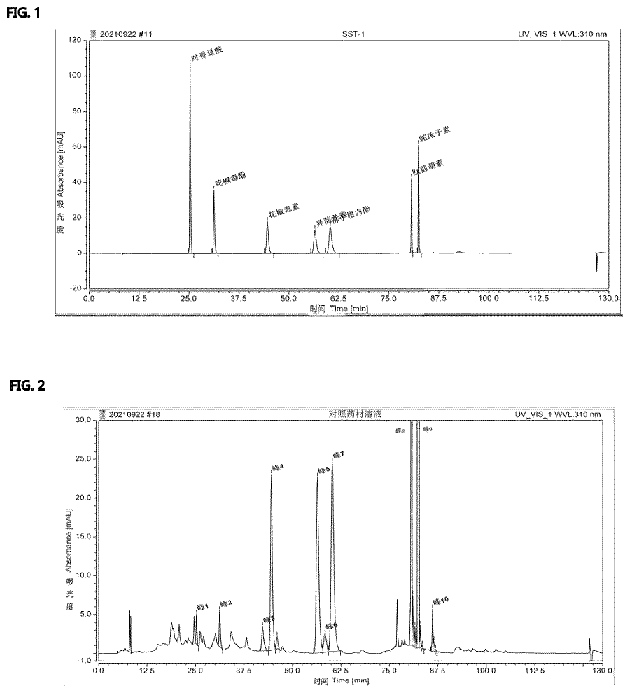

Zanthoxylol toxin, xanthotoxin, bergamot lactone and imperatorin, put them in 10 mL volumetric flasks, add ethanol to dissolve and dilute them to scale, shake well, then accurately weigh 1.0 mL of the above solutions respectively, put them in 10 mL volumetric flasks, dilute them to scale with 70% ethanol, and shake well to get the final product. The spectrum determined by the method of the invention is shown in FIG. 1.

### 4.2.2 Preparation of Samples

4.2.2.1 Preparation of medicinal material sample: Take about 0.5 g of medicinal material, add 50 mL of 70% ethanol accurately, weigh it, and after ultrasonic treatment for 60 min, weigh it again after the solution is cooled to room temperature, make up the lost weight with 70% ethanol, shake it evenly, filter it with microporous membrane, and take the filtrate. The spectrograms determined by the method of the invention are shown in FIG. 2 (Fructus Cnidii as control medicine) and FIG. 3 (Fructus Cnidii as medicine).

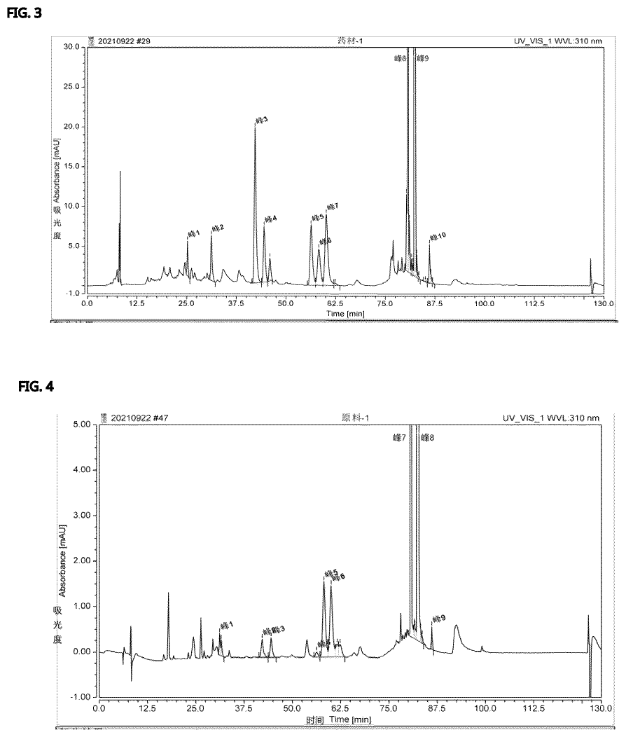

4.2.2.2 Preparation of the extract sample: Accurately weigh about 50 mg of the total coumarin raw material of Fructus Cnidii, accurately add 50 ml of 70% ethanol, weigh it, perform ultrasonic treatment for 60 min, then weigh it again after the solution is cooled to room temperature, make up the lost weight with 70% ethanol, shake it evenly, filter it with a microporous membrane, and take the filtrate. The spectrum determined by the method of the present invention is shown in FIG. 4.

4.2.2.3 Preparation of ointment sample: Accurately weigh about 0.1 g of Ointment of total coumarin of Fructus Cnidii, accurately add 50 ml of 70% ethanol, weigh it, and after ultrasonic treatment for 60 min, weigh it again after the solution is cooled to room temperature, make up the lost weight with 70% ethanol, shake it evenly, filter it with microporous membrane, and take the filtrate. The spectrum determined by the method of the present invention is shown in FIG. 5.

### 5. Methodology Verification

### 5.1 Specificity Test

### 5.1.1 Specificity of Medicinal Materials

Prepare mixed reference solution and sample test solution respectively according to the methods in 4.2.1 and 4.2.2, and prepare blank solution and negative sample (excluding extract) of ointment preparation at the same time, determine according to the determined chromatographic conditions, and record the chromatogram.

### 5.1.2 Test Conclusion

1) The blank solvent did not interfere with the detection of 10 characteristic peaks of Fructus Cnidii, and the retention time was consistent with that of the reference peaks, among which the retention time of p-coumaric acid, Zanthoxylol, xanthotoxin, isoanisidin, bergamot lactone, imperatorin and osthole was consistent with that of the corresponding peaks in the mixed reference solution. It shows that this method has good specificity for the fingerprint determination of Fructus Cnidii.

2) The blank solvent did not interfere with the detection of nine characteristic peaks of total coumarin in Fructus Cnidii, among which p-coumaric acid, Zanthoxylol, xanthotoxin, isoanisidin, bergamot lactone, imperatorin and osthole had the same retention time as the corresponding peaks in the mixed reference solution. It shows that this method has good specificity for the fingerprint determination of total coumarin in Fructus Cnidii.

3) Blank solvents and negative samples did not interfere with the detection of nine characteristic peaks in the total coumarin ointment of Fructus Cnidii, among which p-coumaric acid, zanthoxylol, xanthotoxin, isoanisidin, bergamot lactone, imperatorin and osthole had the same retention time as the corresponding peaks in the mixed reference solution. It shows that this method has good specificity for the fingerprint determination of total coumarin ointment of Fructus Cnidii.

### 5.2 Precision Test

### 5.2.1 Test Method

Take the mixed reference solution, take osthole as reference, determine it according to the selected chromatographic conditions, record the chromatogram, and calculate the relative retention time and relative peak area. The results are shown in Table 4˜5.

TABLE 4
Investigation results of precision of reference solution (relative peak area)

| | p- | | | | | | |
|---|---|---|---|---|---|---|---|
| | coumaric | | | | Bergamot | | |
| Name | acid | Zanthoxylol | Xanthotoxin | Isoanisidin, | lactone | Imperatorin | Osthole |
| SST-1 | 2.437 | 1.067 | 0.917 | 0.899 | 1.148 | 0.672 | 1.000 |
| SST-2 | 2.440 | 1.069 | 0.919 | 0.902 | 1.147 | 0.672 | 1.000 |
| SST-3 | 2.441 | 1.069 | 0.919 | 0.902 | 1.149 | 0.672 | 1.000 |
| SST-4 | 2.442 | 1.069 | 0.918 | 0.902 | 1.148 | 0.672 | 1.000 |
| SST-5 | 2.442 | 1.069 | 0.919 | 0.901 | 1.147 | 0.672 | 1.000 |
| SST-6 | 2.442 | 1.069 | 0.919 | 0.900 | 1.148 | 0.672 | 1.000 |
| RSD (%) | 0.1 | 0.1 | 0.1 | 0.2 | 0.1 | 0.0 | 0.0 |

TABLE 5
Investigation results of precision of reference solution (relative retention time)

| | p- | | | | | | |
|---|---|---|---|---|---|---|---|
| | coumaric | | | | Bergamot | | |
| Name | acid | Zanthoxylol | Xanthotoxin | Isoanisidin, | lactone | Imperatorin | Osthole |
| SST-1 | 0.306 | 0.379 | 0.541 | 0.686 | 0.732 | 0.979 | 1.000 |
| SST-2 | 0.307 | 0.379 | 0.542 | 0.687 | 0.734 | 0.979 | 1.000 |
| SST-3 | 0.307 | 0.379 | 0.542 | 0.687 | 0.734 | 0.979 | 1.000 |
| SST-4 | 0.307 | 0.379 | 0.542 | 0.686 | 0.733 | 0.979 | 1.000 |
| SST-5 | 0.306 | 0.379 | 0.541 | 0.685 | 0.731 | 0.979 | 1.000 |
| SST-6 | 0.306 | 0.379 | 0.541 | 0.685 | 0.732 | 0.979 | 1.000 |
| RSD (%) | 0.2 | 0.0 | 0.2 | 0.2 | 0.2 | 0.0 | 0.0 |

The relative retention time RSD of each characteristic peak in the reference solution is in the range of 0.0% ˜ 0.2%, and the relative peak area RSD is in the range of 0.0% ˜ 0.2%, which indicates that the precision of the instrument is good.

### 5.2.2 Test Conclusion

### 5.3 Repeatability Test

### 5.3.1 Operation Method

Take the same medicinal material, extract and ointment preparation, and prepare 6 test solutions in parallel according to the method under 4.2 Reference substance and sample preparation. Take osthole as a reference, measure it according to the selected chromatographic conditions, record the chromatogram, calculate the relative retention time and relative peak area, and investigate its RSD. The results are shown in Table 6˜Table 11.

TABLE 6
Repeatability of Fructus Cnidii (relative peak area)

| Sample name | Peak 1 | Peak 2 | Peak 3 | Peak 4 | Peak 5 | Peak 6 | Peak 7 | Peak 8 | Peak 9 | Peak 10 |
|---|---|---|---|---|---|---|---|---|---|---|
| Repeatability 1 | 0.00851 | 0.0162 | 0.0790 | 0.0275 | 0.0282 | 0.0292 | 0.0571 | 0.274 | 1.000 | 0.00872 |
| Repeatability 2 | 0.00074 | 0.0152 | 0.0277 | 0.0277 | 0.0405 | 0.0274 | 0.0578 | 0.278 | 1.000 | 0.00 [symbol] |
| Repeatability 3 | 0.00 [symbol] | 0.0151 | 0.0598 | 0.294 | 0.0 [symbol] | 0.0275 | 0.0570 | 0.280 | 1.000 | 0.00 [symbol] |
| Repeatability 4 | 0.00841 | 0.0163 | 0.0780 | 0.6277 | 0.0 [symbol] | 0.0286 | 0.0570 | 0.277 | 1.000 | 0.0 [symbol] |
| Repeatability 5 | 0.0082 [symbol] | 0.0158 | 0.0738 | 0.0281 | 0.0418 | 0.0278 | 0.0583 | 0.270 | 1.000 | 0.00878 |
| Repestability 6 | 0.0084 | 0.0181 | 0.0739 | 0.0789 | 0.0415 | 0.0288 | 0.0572 | 0.278 | 1.000 | 0.00 [symbol] |
| RSD (%) | 2.2 | 3.3 | 5.3 | 2.8 | 1.5 | 2.7 | 0.9 | 0.8 | 0.8 | 0.5 |

[symbol]

indicates data missing or illegible when filed

7 Repeatability of Fructus Cnidii (relative retention time)
T

[symbol]

| Sample name | Peak 1 | Peak 2 | Peak 3 | Peak 4 | Peak 5 | Peak 6 | Peak 7 | Peak 8 | Peak 9 | Peak 10 |
|---|---|---|---|---|---|---|---|---|---|---|
| Repeatability 1 | 0.30 [symbol] | 0.379 | 0.512 | 0.540 | 0.084 | 0.707 | 0.730 | 0.979 | 1.000 | 1.04 [symbol] |
| Repeatability 2 | 0.30 [symbol] | 0.379 | 0.512 | 0.540 | 0.083 | 0.707 | 0.730 | 0.979 | 1.000 | 1.04 [symbol] |
| Repeatability 3 | 0.30 [symbol] | 0.379 | 0.512 | 0.540 | 0.083 | 0.707 | 0.729 | 0.979 | 1.000 | 1.04 [symbol] |
| Repeatability 4 | 0.30 [symbol] | 0.379 | 0.512 | 0.540 | 0.083 | 0.706 | 0.729 | 0.979 | 1.000 | 1.04 [symbol] |
| Repeatability 5 | 0.30 [symbol] | 0.378 | 0.512 | 0.539 | 0.083 | 0.706 | 0.729 | 0.979 | 1.000 | 1.04 [symbol] |
| Repeatability 6 | 0.30 [symbol] | 0.378 | 0.512 | 0.539 | 0.083 | 0.706 | 0.729 | 0.979 | 1.000 | 1.04 [symbol] |
| RSD (%) | 0.0 | 0.2 | 0.0 | 0.1 | 0.1 | 0.1 | 0.1 | 0.0 | 0.0 | 0.0 |

[symbol]

indicates data missing or illegible when filed

Repeatability of Total Coumarin from Fructus Cnidii (relative peak area)

[symbol]

| Sample name | Peak 1 | Peak 2 | Peak 3 | Peak 4 | Peak 5 | Peak 6 | Peak 7 | Peak 8 | Peak 9 |
|---|---|---|---|---|---|---|---|---|---|
| Repeatability 1 | 0.000 [symbol] | 0.000288 | 0.000280 | 0.000 [symbol] | 0.00150 | 0.00144 | 0.0131 | 1.000 | 0.000124 |
| Repeatability 2 | 0.000140 | 0.000255 | 0.000276 | 0.000 [symbol] | 0.00137 | 0.00128 | 0.0128 | 1.000 | 0.000128 |
| Repeatability 3 | 0.000145 | 0.000264 | 0.000 [symbol] | 0.000 [symbol] | 0.00141 | 0.00138 | 0.0132 | 1.000 | 0.000130 |
| Repeatability 4 | 0.000144 | 0.000263 | 0.000273 | 0.000 [symbol] | 0.00141 | 0.00130 | 0.0129 | 1.000 | 0.000125 |
| Repeatability 5 | 0.000149 | 0.000274 | 0.000273 | 0.000 [symbol] | 0.00145 | 0.00133 | 0.0128 | 1.000 | 0.000126 |
| Repeatability 6 | 0.000 [symbol] | 0.000316 | 0.000 [symbol] | 0.000 [symbol] | 0.00160 | 0.00144 | 0.0124 | 1.000 | 0.000 [symbol] |
| RSD (%) | [symbol] | 8.1 | 1.3 | 5.6 | 5.7 | 5.2 | 2.2 | 0.0 | 4.2 |

[symbol]

indicates data missing or illegible when filed

9 Repeatability of Total Coumarin from Fructus Cnidii (Relative Retention Time)

| Sample name | Peak 1 | Peak 2 | Peak 3 | Peak 4 | Peak 5 | Peak 6 | Peak 7 | Peak 8 | Peak 9 |
|---|---|---|---|---|---|---|---|---|---|
| Repeatability 1 | 0.378 | 0.512 | 0.540 | 0. [symbol] | 0.70 [symbol] | 0.729 | 0. [symbol] | 1.00 | 1.045 |
| Repeatability 2 | 0.378 | 0.512 | 0.540 | 0. [symbol] | 0.70 [symbol] | 0.729 | 0. [symbol] | 1.00 | 1.045 |
| Repeatability 3 | 0.378 | 0.512 | 0.540 | 0. [symbol] | 0.70 [symbol] | 0.729 | 0. [symbol] | 1.00 | 1.045 |
| Repeatability 4 | 0.378 | 0.512 | 0.540 | 0. [symbol] | 0.70 [symbol] | 0.729 | 0. [symbol] | 1.00 | 1.045 |
| Repeatability 5 | 0.378 | 0.512 | 0.540 | 0. [symbol] | 0.70 [symbol] | 0.728 | 0. [symbol] | 1.00 | 1.045 |
| Repeatability 6 | 0.378 | 0.512 | 0.540 | 0. [symbol] | 0.70 [symbol] | 0.729 | 0. [symbol] | 1.00 | 1.045 |
| RSD(%) | 0.0 | 0.0 | 0.0 | 0.1 | 0.0 | 0.1 | 0.0 | 0.0 | 0.0 |

[symbol]

indicates data missing or illegible when filed

10 Repeatability Test Results of Total Coumarin Ointment of Fructus Cnidii (Relative Retention Time)

[symbol]

| Sample name | Peak 1 | Peak 2 | Peak 3 | Peak 4 | Peak 5 | Peak 6 | Peak 7 | Peak 8 | Peak 9 |
|---|---|---|---|---|---|---|---|---|---|
| Repeatability 1 | 0.378 | 0.512 | 0.540 | 0. [symbol] | 0.70 [symbol] | 0.729 | 0.979 | 1.00 | 1.04 [symbol] |
| Repeatability 2 | 0.378 | 0.512 | 0.540 | 0. [symbol] | 0.70 [symbol] | 0.730 | 0.979 | 1.00 | 1.04 [symbol] |
| Repeatability 3 | 0.378 | 0.512 | 0.540 | 0. [symbol] | 0.70 [symbol] | 0.729 | 0. [symbol] | 1.00 | 1.04 [symbol] |
| Repeatability 4 | 0.378 | 0.512 | 0.540 | 0. [symbol] | 0.70 [symbol] | 0.730 | 0.979 | 1.00 | 1.04 [symbol] |
| Repeatability 5 | 0.379 | 0.513 | 0.541 | 0. [symbol] | 0.70 [symbol] | 0.732 | 0. [symbol] | 1.00 | 1.04 [symbol] |
| Repeatability 6 | 0.379 | 0.513 | 0.541 | 0. [symbol] | 0.70 [symbol] | 0.732 | 0. [symbol] | 1.00 | 1.04 [symbol] |
| RSD (%) | 0.2 | 0.2 | 0.1 | 0.2 | 0.2 | 0.2 | 0.0 | 0.0 | 0.0 |

[symbol]

indicates data missing or illegible when filed

1 Repeatability test results of total coumarin ointment of Fructus Cnidii (relative peak area)

| Sample name | Peak 1 | Peak 2 | Peak 3 | Peak 4 | Peak 5 | Peak 6 | Peak 7 | Peak 8 | Peak 9 |
|---|---|---|---|---|---|---|---|---|---|
| Repeatability 1 | 0.0004 [symbol] | 0.000223 | 0.00175 | 0.0004 [symbol] | 0.0011 [symbol] | 0.00 [symbol] | 0.07 [symbol] | 1.000 | 0.001 [symbol] |
| Repeatability 2 | 0.0004 [symbol] | 0.000269 | 0.00174 | 0.0004 [symbol] | 0.0011 [symbol] | 0.00 [symbol] | 0.07 [symbol] | 1.000 | 0.001 [symbol] |
| Repeatability 3 | 0.0004 [symbol] | 0.0002 [symbol] | 0.0017 [symbol] | 0.0004 [symbol] | 0.0011 [symbol] | 0.00 [symbol] | 0.07 [symbol] | 1.000 | 0.001 [symbol] |
| Repeatability 4 | 0.0004 [symbol] | 0.000 [symbol] | 0.00177 | 0.0004 [symbol] | 0.0011 [symbol] | 0.00 [symbol] | 0.07 [symbol] | 1.000 | 0.001 [symbol] |
| Repeatability 5 | 0.0004 [symbol] | 0.000 [symbol] | 0.00175 | 0.0004 [symbol] | 0.0011 [symbol] | 0.00 [symbol] | 0.07 [symbol] | 1.000 | 0.001 [symbol] |
| Repeatability 6 | 0.0004 [symbol] | 0.000193 | 0.00171 | 0.0004 [symbol] | 0.0011 [symbol] | 0.00 [symbol] | 0.07 [symbol] | 1.000 | 0.001 [symbol] |
| RSD (%) | 1.2 | 12.8 | 1.2 | 1.4 | 1.4 | 1. [symbol] | 1. [symbol] | 0.0 | 0.0 |

[symbol]

indicates data missing or illegible when filed

### Test Conclusion

(1) Six samples of Fructus Cnidii were determined repeatedly, and the relative peak area RSD of each characteristic peak was in the range of 0. 8% ˜ 5.3%, and the relative retention time RSD was in the range of 0.0% ˜ 0.2%, which indicated that the characteristic map analysis method had good repeatability.

(2) Six samples of total coumarin in Fructus Cnidii were repeatedly determined, and the relative retention time RSD of each characteristic peak was in the range of 0. 0% ˜ 0.1%, and the relative peak area RSD was in the range of 1.3% ˜ 8.1%, which indicated that the characteristic map analysis method had good repeatability.

(3) Six samples of Fructus Cnidii total coumarin ointment were repeatedly determined, and the relative retention time RSD of each characteristic peak was in the range of 0. 0% ˜ 0.2%, and the relative peak area RSD was in the range of 0.8% ˜ 12.8%, which indicated that the characteristic map analysis method had good repeatability.

### 5. 4 Intermediate Precision

### 5.4.1 Operation Method

Other analysts operate on different dates, chromatographs and chromatographic columns with different batch numbers, and take the same batch of samples of Fructus Cnidii, extracts and ointments, and prepare 6 test solutions in parallel according to the method under 4.2 Reference substance and sample preparation. Taking Fructus Cnidii as a reference, determine the samples according to the selected chromatographic conditions, record the chromatogram, and calculate the relative retention time and relative peak area. The experimental results are shown in Table 12-Table 17.

TABLE 12
Investigation results of intermediate precision of Fructus Cnidii (relative peak area)

| sample name | Peak 1 | Peak 2 | Peak 3 | Peak 4 | Peak 5 | Peak 6 | Peak 7 | Peak 8 | Peak 9 | Peak 10 |
|---|---|---|---|---|---|---|---|---|---|---|
| Intermediate | 0.0115 | 0.0160 | 0.0710 | 0.0306 | 0.0409 | 0.0279 | 0.0542 | 0.265 | 1.0000 | 0.00753 |
| precision 1 | | | | | | | | | | |
| Intermediate | 0.0119 | 0.0157 | 0.0680 | 0.0299 | 0.0404 | 0.0268 | 0.0542 | 0.266 | 1.0000 | 0.00750 |
| precision 2 | | | | | | | | | | |
| Intermediate | 0.0114 | 0.0160 | 0.0691 | 0.0306 | 0.0412 | 0.0277 | 0.0532 | 0.266 | 1.0000 | 0.00741 |
| precision 3 | | | | | | | | | | |
| Intermediate | 0.0116 | 0.0158 | 0.0696 | 0.0310 | 0.0410 | 0.0279 | 0.0539 | 0.266 | 1.0000 | 0.00752 |
| precision 4 | | | | | | | | | | |
| Intermediate | 0.0103 | 0.0127 | 0.0535 | 0.031 [symbol] | 0.0423 | 0.0226 | 0.0577 | 0.265 | 1.0000 | 0.00704 |
| precision 5 | | | | | | | | | | |
| Intermediate | 0.011 [symbol] | 0.0143 | 0.0594 | 0.0302 | 0.0412 | 0.0247 | 0.0561 | 0.266 | 1.0000 | 0.00724 |
| precision 6 | | | | | | | | | | |
| RSD(%) | 5.0 | 8.9 | 10.8 | 1.5 | 1.6 | 8.3 | 3.1 | 0.2 | 0.0 | 2.7 |
| And repeatability | 16.2 | 6.7 | 30.4 | 4.6 | 1.5 | 6.8 | 3.2 | 2.3 | 0.0 | 8.7 |
| RSD (%) | | | | | | | | | | |

[symbol]

indicates data missing or illegible when filed

TABLE 13
Investigation results of intermediate precision of Fructus Cnidii (relative retention time)

| Sample name | Peak 1 | Peak 2 | Peak 3 | Peak 4 | Peak 5 | Peak 6 | Peak 7 | Peak 8 | Peak 9 | Peak 10 |
|---|---|---|---|---|---|---|---|---|---|---|
| Intermediate | 0.308 | 0.386 | 0.532 | 0.560 | 0.717 | 0.746 | 0.766 | 0.979 | 1.000 | 1.049 |
| precision 1 | | | | | | | | | | |
| Intermediate | 0.308 | 0.386 | 0.531 | 0.560 | 0.716 | 0.745 | 0.765 | 0.979 | 1.000 | 1.049 |
| precision 2 | | | | | | | | | | |
| Intermediate | 0.308 | 0.386 | 0.531 | 0.560 | 0.716 | 0.745 | 0.766 | 0.979 | 1.000 | 1.049 |
| precision 3 | | | | | | | | | | |
| Intermediate | 0.308 | 0.386 | 0.531 | 0.560 | 0.716 | 0.745 | 0.765 | 0.979 | 1.000 | 1.049 |
| precision 4 | | | | | | | | | | |
| Intermediate | 0.308 | 0.386 | 0.531 | 0.560 | 0.716 | 0.745 | 0.765 | 0.979 | 1.000 | 1.049 |
| precision 5 | | | | | | | | | | |
| Intermediate | 0.308 | 0.386 | 0.531 | 0.559 | 0.716 | 0.745 | 0.765 | 0.979 | 1.000 | 1.049 |
| precision 6 | | | | | | | | | | |
| RSD(%) | 0.0 | 0.1 | 0.1 | 0.1 | 0.1 | 0.1 | 0.1 | 0.0 | 0.0 | 0.1 |
| And repeatability | 0.4 | 1.1 | 2.0 | 2.0 | 2.5 | 2.8 | 2.6 | 0.1 | 0.0 | 0.2 |
| RSD (%) | | | | | | | | | | |

TABLE 14
Investigation results of intermediate precision (relative peak area) of total coumarin in Fructus Cnidii.

| Sample Name | Peak 1 | Peak 2 | Peak 3 | Peak 4 | Peak 5 | Peak 6 | Peak 7 | Peak 8 | Peak 9 |
|---|---|---|---|---|---|---|---|---|---|
| Intermediate | 0.000226 | 0.000334 | 0.000371 | 0.0000570 | 0.00195 | 0.00156 | 0.0175 | 1.0000 | 0.000171 |
| precision 2 | | | | | | | | | |
| Intermediate | 0.000237 | 0.000361 | 0.000372 | 0.0000580 | 0.00200 | 0.00153 | 0.0180 | 1.0000 | 0.000333 |
| precision 3 | | | | | | | | | |
| Intermediate | 0.000232 | 0.000338 | 0.000364 | 0.0000520 | 0.00196 | 0.00155 | 0.0176 | 1.0000 | 0.000173 |
| precision 4 | | | | | | | | | |
| Intermediate | 0.000230 | 0.000339 | 0.000361 | 0.0000510 | 0.00197 | 0.00155 | 0.0177 | 1.0000 | 0.000163 |
| precision 5 | | | | | | | | | |
| Intermediate | 0.000250 | 0.000360 | 0.000388 | 0.0000620 | 0.00211 | 0.00166 | 0.0190 | 1.0000 | 0.000187 |
| precision 6 | | | | | | | | | |
| RSD(%) | 3.9 | 3.7 | 2.6 | 7.9 | 3.3 | 3.3 | 3.4 | 0.0 | 4.6 |
| And repeatability | 23.6 | 13.0 | 16.0 | 26.4 | 16.6 | 8.6 | 17.3 | 0.0 | 17.5 |
| RSD%% | | | | | | | | | |

TABLE 15
Investigation results of intermediate precision of total
coumarin in Fructus Cnidii (relative retention time)

| sample name | Peak 1 | Peak 2 | Peak 3 | Peak 4 | Peak 5 | Peak 6 | Peak 7 | Peak 8 | Peak 9 |
|---|---|---|---|---|---|---|---|---|---|
| Intermediate | 0.3855 | 0.5322 | 0.5602 | 0.7174 | 0.7462 | 0.7698 | 0.9795 | 1.0000 | 1.0486 |
| precision 1 | | | | | | | | | |
| Intermediate | 0.3854 | 0.5323 | 0.5603 | 0.7168 | 0.7461 | 0.7698 | 0.9795 | 1.0000 | 1.0485 |
| precision 2 | | | | | | | | | |
| Intermediate | 0.3854 | 0.5320 | 0.5599 | 0.7160 | 0.7457 | 0.7695 | 0.9795 | 1.0000 | 1.0485 |
| precision 3 | | | | | | | | | |
| Intermediate | 0.3854 | 0.5319 | 0.5601 | 0.7161 | 0.7453 | 0.7680 | 0.9794 | 1.0000 | 1.0485 |
| precision 4 | | | | | | | | | |
| Intermediate | 0.3855 | 0.5321 | 0.5600 | 0.7164 | 0.7457 | 0.7689 | 0.9793 | 1.0000 | 1.0485 |
| precision 5 | | | | | | | | | |
| Intermediate | 0.3857 | 0.5321 | 0.5602 | 0.7178 | 0.7456 | 0.7692 | 0.9794 | 1.0000 | 1.0486 |
| precision 6 | | | | | | | | | |
| RSD(%) | 0.1 | 0.1 | 0.1 | 0.2 | 0.1 | 0.1 | 0.1 | 0.0 | 0.1 |
| And repeatability | 1.0 | 2.1 | 1.9 | 2.5 | 2.9 | 2.9 | 0.1 | 0.0 | 0.2 |
| RSD (%) | | | | | | | | | |

TABLE 16
Intermediate precision test results (relative peak area) of Ointment of total coumarin of Fructus Cnidii

| sample name | Peak 1 | Peak 2 | Peak 3 | Peak 4 | Peak 5 | Peak 6 | Peak 7 | Peak 8 | Peak 9 |
|---|---|---|---|---|---|---|---|---|---|
| Intermediate | 0.000388 | 0.000259 | 0.00182 | 0.000442 | 0.00120 | 0.00409 | 0.0762 | 1.0000 | 0.00136 |
| precision 1 | | | | | | | | | |
| Intermediate | 0.000415 | 0.000254 | 0.00185 | 0.000461 | 0.00118 | 0.00430 | 0.0783 | 1.0000 | 0.00137 |
| precision 2 | | | | | | | | | |
| Intermediate | 0.000415 | 0.000249 | 0.00177 | 0.000478 | 0.00132 | 0.00434 | 0.0743 | 1.0000 | 0.00135 |
| precision 3 | | | | | | | | | |
| Intermediate | 0.000403 | 0.000284 | 0.00182 | 0.000447 | 0.00123 | 0.00422 | 0.0790 | 1.0000 | 0.00144 |
| precision 4 | | | | | | | | | |
| Intermediate | 0.000389 | 0.000248 | 0.00175 | 0.000453 | 0.00123 | 0.00424 | 0.0787 | 1.0000 | 0.00144 |
| precision 5 | | | | | | | | | |
| Intermediate | 0.000385 | 0.000194 | 0.00176 | 0.000439 | 0.00123 | 0.00425 | 0.0755 | 1.0000 | 0.00134 |
| precision 6 | | | | | | | | | |
| RSD(%) | 3.5 | 12.0 | 2.3 | 3.2 | 3.9 | 2.1 | 2.6 | 0.0 | 3.3 |
| And repeatability | 8.1 | 16.5 | 2.3 | 6.3 | 7.1 | 7.1 | 2.0 | 0.0 | 3.8 |
| RSD %% | | | | | | | | | |

TABLE 17
Intermediate precision test results of total coumarin
ointment of Fructus Cnidii (relative retention time)

| Sample name | Peak 1 | Peak 2 | Peak 3 | Peak 4 | Peak 5 | Peak 6 | Peak 7 | Peak 8 | Peak 9 |
|---|---|---|---|---|---|---|---|---|---|
| Intermediate | 0.386 | 0.533 | 0.561 | 0.717 | 0.747 | 0.768 | 0.979 | 1.000 | 1.048 |
| precision 1 | | | | | | | | | |
| Intermediate | 0.386 | 0.532 | 0.561 | 0.717 | 0.747 | 0.768 | 0.979 | 1.000 | 1.049 |
| precision 2 | | | | | | | | | |
| Intermediate | 0.386 | 0.532 | 0.560 | 0.717 | 0.747 | 0.768 | 0.979 | 1.000 | 1.048 |
| precision 3 | | | | | | | | | |
| Intermediate | 0.386 | 0.532 | 0.560 | 0.718 | 0.747 | 0.768 | 0.979 | 1.000 | 1.048 |
| precision 4 | | | | | | | | | |
| Intermediate | 0.386 | 0.532 | 0.560 | 0.717 | 0.746 | 0.767 | 0.979 | 1.000 | 1.048 |
| precision 5 | | | | | | | | | |
| Intermediate | 0.386 | 0.532 | 0.560 | 0.716 | 0.747 | 0.768 | 0.979 | 1.000 | 1.048 |
| precision 6 | | | | | | | | | |
| RSD(%) | 0.1 | 0.1 | 0.1 | 0.1 | 0.1 | 0.1 | 0.0 | 0.0 | 0.1 |
| And repeatability | 1.1 | 2.0 | 1.9 | 2.5 | 2.9 | 2.7 | 0.1 | 0.0 | 0.2 |
| RSD (%) | | | | | | | | | |

### 5.4.2 Test Conclusion

(1) The relative retention time of 10 common characteristic peaks was in the range of 0.0%-0.1%, and the relative peak area of 10 common characteristic peaks was in the range of 0.1%-2.8% compared with 6 data of repeatability test.

(2) Different analysts operated on different dates, chromatographs and chromatographic columns with different batch numbers. The relative retention time of nine common characteristic peaks in the total coumarin raw material of Fructus Cnidii was in the range of 0. 1% ˜ 0.2%, and the relative peak area of nine common characteristic peaks was in the range of 0.1% ˜ 2.9% compared with the six data of repeatability test.

(3) Different analysts operated on different dates, chromatographs and chromatographic columns with different batch numbers. The relative retention time of nine common characteristic peaks in Fructus Cnidii total coumarins ointment was in the range of 0. 0% ˜ 0.1%, and the relative peak area of the nine common characteristic peaks was in the range of 0.1% ˜ 2.9% compared with the six data of repeated tests.

### 5.5 Stability Test

### 5.5.1 Test Method

According to the method under item 4.2, the sample solutions of medicinal materials, extracts and ointments were prepared, placed at room temperature, and injected at 0, 21, 40, 60, 87 and 94 hours respectively. The results are shown in Table 18˜Table 23.

TABLE 18
Investigation results of stability of test solution of Fructus Cnidii (relative peak area)

| time | Peak 1 | Peak 2 | Peak 3 | Peak 4 | Peak 5 | Peak 6 | Peak 7 | Peak 8 | Peak 9 | Peak 10 |
|---|---|---|---|---|---|---|---|---|---|---|
| 0 h | 0.00862 | 0.0161 | 0.0758 | 0.0273 | 0.0404 | 0.0286 | 0.0566 | 0.276 | 1.00 | 0.00882 |
| 21 h | 0.00850 | 0.0164 | 0.0757 | 0.0271 | 0.0404 | 0.0286 | 0.0566 | 0.277 | 1.00 | 0.00882 |
| 40 h | 0.00853 | 0.0164 | 0.0758 | 0.0270 | 0.0402 | 0.0286 | 0.0566 | 0.276 | 1.00 | 0.00878 |
| 60 h | 0.00858 | 0.0164 | 0.0758 | 0.0272 | 0.0403 | 0.0286 | 0.0565 | 0.276 | 1.00 | 0.00884 |
| 87 h | 0.00823 | 0.0164 | 0.0764 | 0.0271 | 0.0396 | 0.0284 | 0.0561 | 0.269 | 1.00 | 0.00861 |
| 94 h | 0.00817 | 0.0165 | 0.0766 | 0.0273 | 0.0395 | 0.0283 | 0.0559 | 0.269 | 1.00 | 0.00857 |
| RSD(%) | 2.3 | 0.9 | 0.6 | 0.5 | 1.1 | 0.5 | 0.6 | 1.4 | 0.0 | 1.4 |

TABLE 19
Investigation results of stability of test solution of Fructus Cnidii (relative retention time)

| time | Peak 1 | Peak 2 | Peak 3 | Peak 4 | Peak 5 | Peak 6 | Peak 7 | Peak 8 | Peak 9 | Peak 10 |
|---|---|---|---|---|---|---|---|---|---|---|
| 0 h | 0.306 | 0.379 | 0.513 | 0.541 | 0.685 | 0.709 | 0.732 | 0.979 | 1.00 | 1.045 |
| 21 h | 0.306 | 0.379 | 0.512 | 0.540 | 0.684 | 0.707 | 0.730 | 0.979 | 1.00 | 1.045 |
| 40 h | 0.306 | 0.378 | 0.512 | 0.540 | 0.683 | 0.706 | 0.729 | 0.979 | 1.00 | 1.045 |
| 60 h | 0.306 | 0.379 | 0.513 | 0.541 | 0.685 | 0.708 | 0.731 | 0.979 | 1.00 | 1.045 |
| 87 h | 0.305 | 0.378 | 0.511 | 0.539 | 0.681 | 0.705 | 0.727 | 0.979 | 1.00 | 1.045 |
| 94 h | 0.305 | 0.378 | 0.511 | 0.538 | 0.681 | 0.705 | 0.727 | 0.979 | 1.00 | 1.045 |
| RSD(%) | 0.2 | 0.2 | 0.2 | 0.3 | 0.3 | 0.3 | 0.3 | 0.0 | 0.0 | 0.0 |

TABLE 20
Investigation results of stability of raw materials of
total coumarin from Fructus Cnidii (relative peak area)

| Time | Peak 1 | Peak 2 | Peak 3 | Peak 4 | Peak 5 | Peak 6 | Peak 7 | Peak 8 | Peak 9 |
|---|---|---|---|---|---|---|---|---|---|
| 0 h | 0.000118 | 0.000211 | 0.000284 | 0.0000785 | 0.00116 | 0.00101 | 0.0139 | 1.000 | 0.000148 |
| 21 h | 0.000119 | 0.000203 | 0.000271 | 0.0000835 | 0.00117 | 0.00102 | 0.0139 | 1.000 | 0.000145 |
| 40 h | 0.000121 | 0.000197 | 0.000278 | 0.0000830 | 0.00118 | 0.00102 | 0.0138 | 1.000 | 0.000147 |
| 60 h | 0.000121 | 0.000204 | 0.000272 | 0.0000813 | 0.00117 | 0.00100 | 0.0137 | 1.000 | 0.000145 |
| 87 h | 0.000120 | 0.000209 | 0.000268 | 0.0000819 | 0.00118 | 0.00102 | 0.0134 | 1.000 | 0.000153 |
| 94 h | 0.000123 | 0.000204 | 0.000279 | 0.0000850 | 0.00118 | 0.00102 | 0.0135 | 1.000 | 0.000148 |
| RSD(%) | 1.5 | 2.5 | 2.2 | 2.6 | 0.7 | 0.9 | 1.6 | 0.0 | 2.0 |

TABLE 21
Investigation results of stability of raw materials of total
coumarin from Fructus Cnidii (relative retention time)

| Time | Peak 1 | Peak 2 | Peak 3 | Peak 4 | Peak 5 | Peak 6 | Peak 7 | Peak 8 | Peak 9 |
|---|---|---|---|---|---|---|---|---|---|
| 0 h | 0.379 | 0.513 | 0.541 | 0.685 | 0.708 | 0.731 | 0.979 | 1.000 | 1.045 |
| 21 h | 0.378 | 0.512 | 0.540 | 0.683 | 0.706 | 0.729 | 0.979 | 1.000 | 1.045 |
| 40 h | 0.378 | 0.512 | 0.540 | 0.683 | 0.706 | 0.729 | 0.979 | 1.000 | 1.045 |
| 60 h | 0.378 | 0.512 | 0.540 | 0.684 | 0.706 | 0.729 | 0.979 | 1.000 | 1.045 |
| 87 h | 0.377 | 0.511 | 0.538 | 0.681 | 0.704 | 0.726 | 0.979 | 1.000 | 1.045 |
| 94 h | 0.377 | 0.511 | 0.538 | 0.680 | 0.704 | 0.726 | 0.979 | 1.000 | 1.045 |
| RSD(%) | 0.2 | 0.2 | 0.3 | 0.3 | 0.3 | 0.3 | 0.0 | 0.0 | 0.0 |

TABLE 22
Stability test results of total coumarin ointment of Fructus Cnidii (relative peak area)

| Time | Peak 1 | Peak 2 | Peak 3 | Peak 4 | Peak 5 | Peak 6 | Peak 7 | Peak 8 | Peak 9 |
|---|---|---|---|---|---|---|---|---|---|
| 0 h | 0.000467 | 0.000264 | 0.00184 | 0.000460 | 0.00122 | 0.00418 | 0.0752 | 1.000 | 0.00148 |
| 21 h | 0.000482 | 0.000253 | 0.00179 | 0.000477 | 0.00123 | 0.00420 | 0.0752 | 1.000 | 0.00144 |
| 40 h | 0.000466 | 0.000226 | 0.00178 | 0.000486 | 0.00125 | 0.00417 | 0.0753 | 1.000 | 0.00145 |
| 60 h | 0.000493 | 0.000259 | 0.00181 | 0.000489 | 0.00126 | 0.00449 | 0.0752 | 1.000 | 0.00147 |
| 87 h | 0.000465 | 0.000272 | 0.00177 | 0.000481 | 0.00124 | 0.00418 | 0.0733 | 1.000 | 0.00141 |
| 94 h | 0.000399 | 0.000274 | 0.00174 | 0.000458 | 0.00126 | 0.00419 | 0.0731 | 1.000 | 0.00142 |
| RSD(%) | 7.2 | 6.8 | 2.0 | 2.8 | 1.4 | 3.0 | 1.5 | 0.0 | 1.9 |

TABLE 23
Stability test results of Ointment of total coumarin of Fructus Cnidii (relative retention time)

| time | Peak 1 | Peak 2 | Peak 3 | Peak 4 | Peak 5 | Peak 6 | Peak 7 | Peak 8 | Peak 9 |
|---|---|---|---|---|---|---|---|---|---|
| 0 h | 0.379 | 0.513 | 0.541 | 0.685 | 0.708 | 0.732 | 0.979 | 1.00 | 1.045 |
| 21 h | 0.378 | 0.513 | 0.540 | 0.684 | 0.706 | 0.730 | 0.979 | 1.00 | 1.045 |
| 40 h | 0.378 | 0.512 | 0.540 | 0.683 | 0.706 | 0.729 | 0.979 | 1.00 | 1.045 |
| 60 h | 0.379 | 0.513 | 0.541 | 0.684 | 0.707 | 0.731 | 0.979 | 1.00 | 1.045 |
| 87 h | 0.377 | 0.511 | 0.538 | 0.681 | 0.704 | 0.727 | 0.979 | 1.00 | 1.045 |
| 94 h | 0.377 | 0.510 | 0.538 | 0.680 | 0.703 | 0.726 | 0.979 | 1.00 | 1.045 |
| RSD(%) | 0.3 | 0.3 | 0.3 | 0.3 | 0.3 | 0.4 | 0.0 | 0.0 | 0.0 |

### 5.5.2 Test Conclusions

(1) Within 94 hours at room temperature, the relative retention time RSD of 10 common characteristic peaks in the test solution of Fructus Cnidii was in the range of 0. 0% ˜ 0.3%, and the relative peak area RSD was in the range of 0.5% ˜ 2.3%, which indicated that the test solution was stable within 94 hours at room temperature.

(2) Within 94 hours at room temperature, the relative retention time RSD of nine common characteristic peaks in the test solution of Fructus Cnidii total coumarin was in the range of 0. 0% ˜ 0.3%, and the relative peak area RSD was in the range of 0.7% ˜ 2.6%, which indicated that the test solution was stable within 94 hours at room temperature.

(3) Within 94 hours at room temperature, the relative retention time RSD of nine common characteristic peaks in the test solution of Ointment of total coumarin of Fructus Cnidii was in the range of 0. 0% ˜ 0.4%, and the relative peak area RSD was in the range of 1.4% ˜ 7.2%, which indicated that the test solution was stable within 94 hours at room temperature.

### 6. Establishment and Analysis of Sample Fingerprint

### 6.1 Establishment of Fingerprint and Similarity Evaluation

Fingerprint analysis was carried out by using “Similarity Evaluation System of Chromatographic Fingerprint of Traditional Chinese Medicine (2012 Edition)”, and data of multiple batches of samples (not less than 15 batches) were collected, and automatic matching was carried out after multi-point correction, and the control fingerprint was generated by median method.

### 6.2 Identification and Attribution of Common Peaks in Fingerprint

(1) Full-scan data acquisition by high-resolution mass spectrometry of liquid chromatography: prepare Fructus Cnidii medicinal materials, Fructus Cnidii total extract, Fructus Cnidii preparation and Fructus Cnidii preparation negative control (except Fructus Cnidii, take other auxiliary materials), sample injection by the same liquid-phase method as the above fingerprint development, and perform full-scan analysis by high-resolution mass spectrometry of liquid chromatography. And use

The similarity evaluation software of chromatographic fingerprints of traditional Chinese medicine identifies the common peaks of 15 batches of characteristic fingerprints of Fructus Cnidii. Taking Fructus Cnidii as the reference peak (S peak), the common peak with stable relative retention time and relative peak area is selected as the characteristic peak.

(2) Component confirmation: Combining the source data of the sample, the characteristic components obtained in the above software are extracted and confirmed.

(3) Speculation and confirmation of chemical structural formula of components: The unique chemical components of Fructus Cnidii (medicinal materials, extracts, preparations) were analyzed, assigned and confirmed by using proprietary Chinese medicine component database and software.

(4) Form a summary report on the chemical composition of Fructus Cnidii (medicinal materials, extracts and preparations) and establish a standard fingerprint.

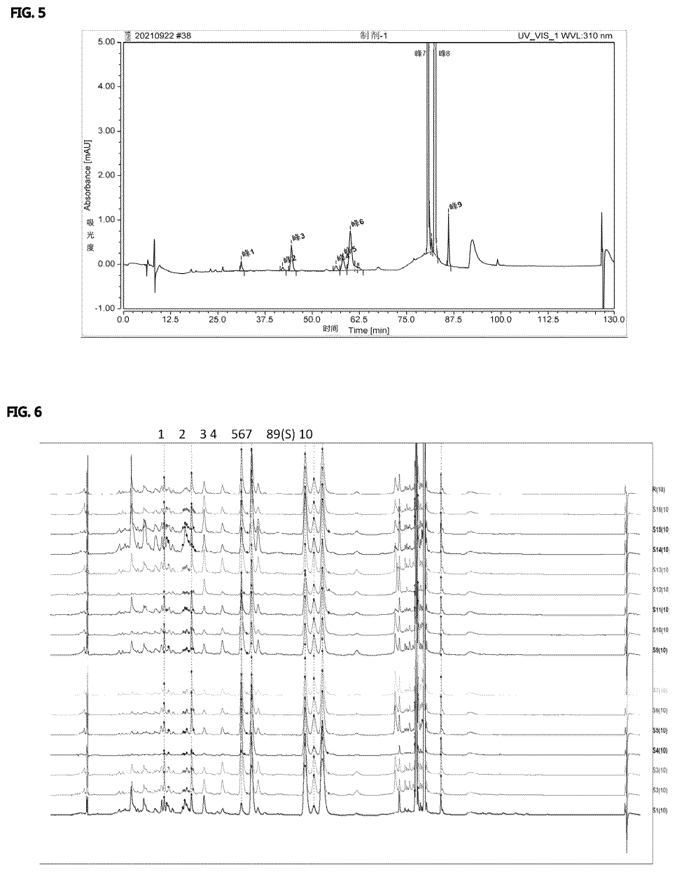

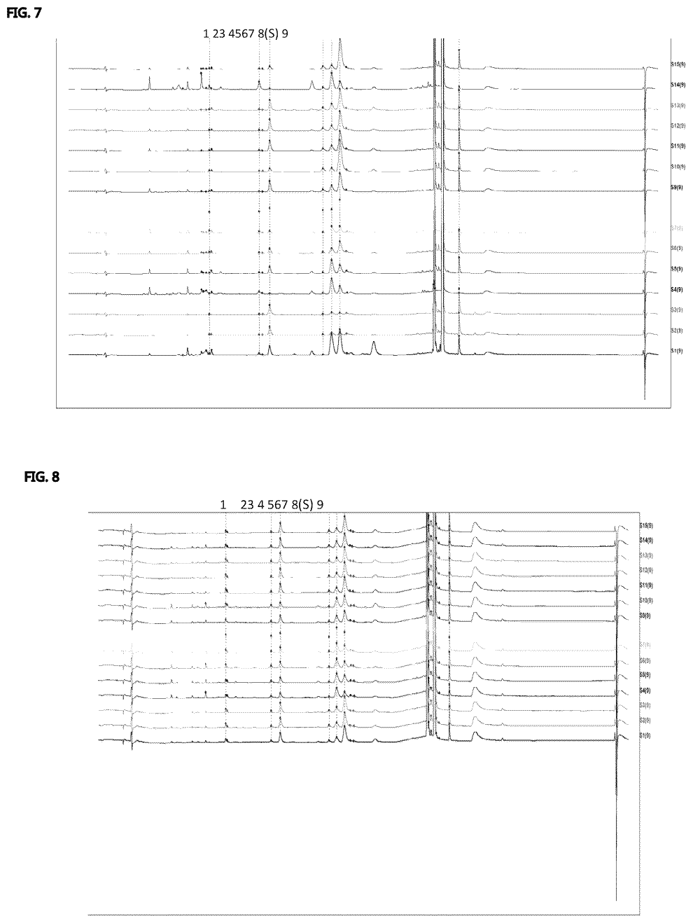

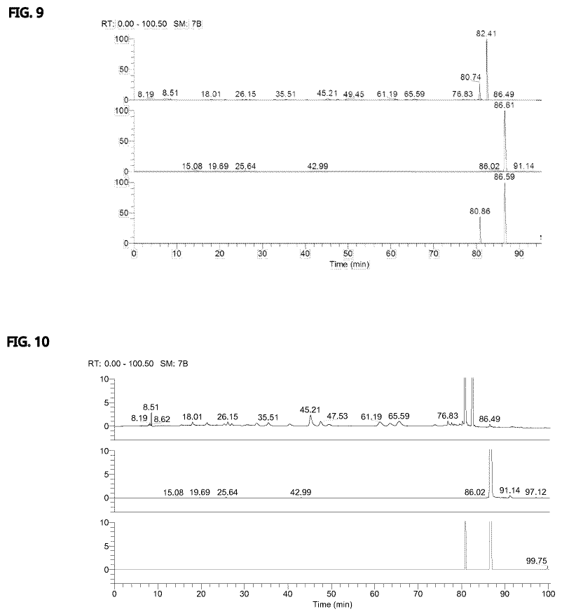

Conclusion: The Chinese medicinal materials Fructus Cnidii and the reference medicinal materials in this invention all meet the quality standards of the current edition of China Pharmacopoeia, and the preparation and inspection of the raw materials of Fructus Cnidii total coumarin and the ointment of Fructus Cnidii total coumarin are carried out according to the national GMP for drug production. In order to investigate the stability of the product and improve the quality standard of the product, the fingerprint of Fructus Cnidii, the total extract of Fructus Cnidii (total coumarin of Fructus Cnidii) and the total coumarin ointment of Fructus Cnidii was studied, and 15 samples of different batches were extracted and determined according to the optimized method. Seven representative common peaks were screened out from the fingerprint and identified, and the separation degree between the peaks was good, and the similarity of the fingerprint between the 15 batches was greater than 90%, indicating that. The characteristic fingerprints of 15 batches of medicinal materials are shown in FIG. 6. The fingerprint of 15 batches of Fructus Cnidii extract (total coumarin of Fructus Cnidii) is shown in FIG. 7. The fingerprint of the preparation (Ointment of total coumarin of Fructus Cnidii) made of 15 batches of Fructus Cnidii extract (Fructus Cnidii Total Coumarin) is shown in FIG. 8. See FIG. 9 to FIG. 14 for the related identification of the unknown peak (No. 10 peak) of Fructus Cnidii.

(1) Through qualitative analysis by high-resolution mass spectrometry, comparison with common characteristic peaks in the characteristic map of Fructus Cnidii, and selection of multiple reference substances for location and identification, it was determined that the characteristic map of Fructus Cnidii contains peak 1: p-coumaric acid, peak 2: zanthoxylol, peak 3: hesperidin hydrate, peak 4: xanthotoxin, peak 5: isoanisidine, peak 6: hesperidone, peak 7: bergamot lactone, peak 8: imperatorin, peak 9: osthole and Peak 10: bergamot.

(2) Through qualitative analysis by high-resolution mass spectrometry, comparison with the common characteristic peaks in the characteristic map of total coumarin raw materials of Fructus Cnidii, and selection of multiple reference substances for location and identification, it was determined that the characteristic map of total coumarin raw materials of Fructus Cnidii contained peak 1: zanthoxylol, peak 2: hesperidin hydrate, peak 3: xanthotoxin, peak 4: isoanisidine, peak 5: hesperidone, peak 6: bergamot lactone and peak 7: imperatorin, peak 8: osthole and Peak 9: bergamot.

(3) Through qualitative analysis by high-resolution mass spectrometry, comparison with the common characteristic peaks in the characteristic map of total coumarin ointment of Fructus Cnidii, and selection of multiple reference substances for location and identification, it was determined that the characteristic map of total coumarin raw materials of Fructus Cnidii contained peak 1: zanthoxylol, peak 2: hesperidin hydrate, peak 3: xanthotoxin, peak 4: isoanisidine, peak 5: hesperidone, peak 6: bergamot lactone and peak 7: imperatorin, peak 8: osthole and Peak 9: bergamot.

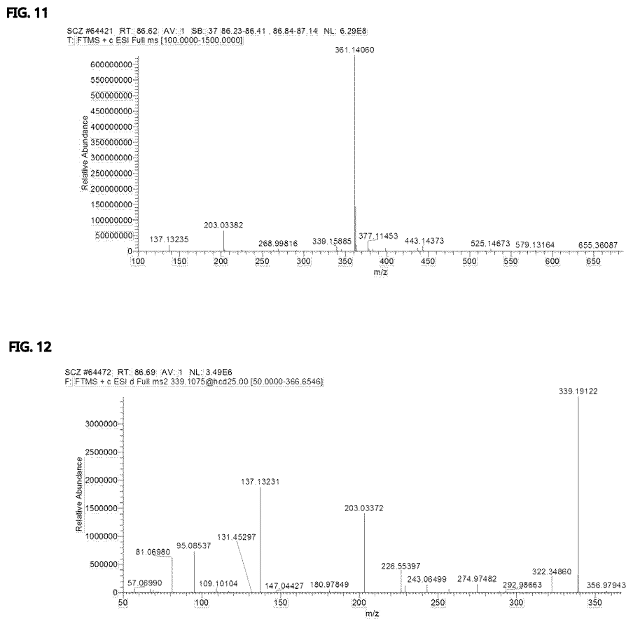

(4) The unknown chromatographic peak No. 10 with retention time of 86. 49 minutes was identified by qualitative analysis of high-resolution mass spectrometry and comparison with the common characteristic peaks in the characteristic map of Fructus Cnidii. In the UV chromatogram, the corresponding first-order mass spectrogram and extracted ion current diagram are taken, as shown in FIG. 11 and FIG. 12. The mass-to-charge ratios of the first-order mass spectrum of the chromatographic peak are 361.14060, 339.15885 and 203.03382, in which 339 and 361 ions are [M+H] and [M+Na] ions of the compound respectively, and 203 is the main fragment ion. The molecular formula of the compound was calculated as C21H2204. See FIG. 11 for its mass spectrum. The secondary mass spectra of the corresponding compounds are extracted, as shown in FIG. 12.

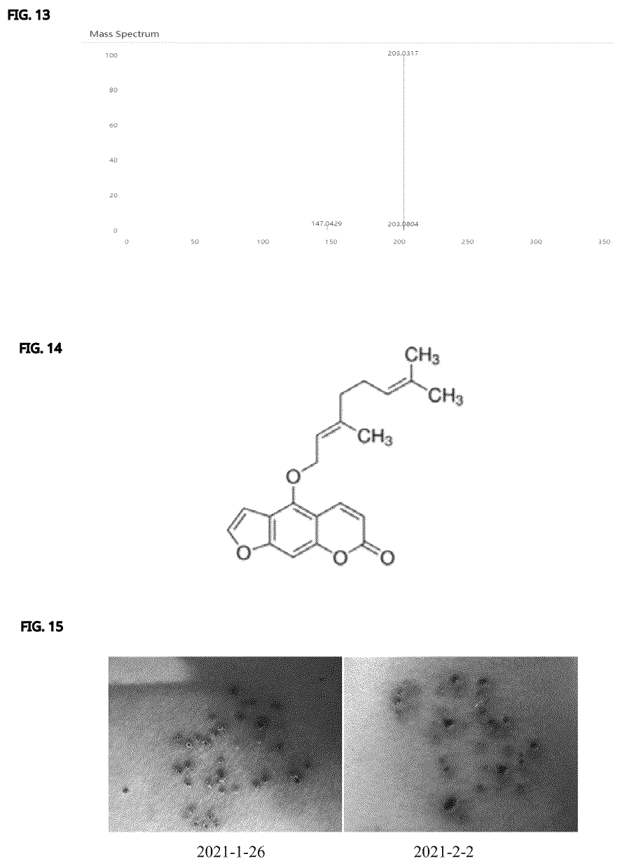

Comparing the secondary spectrum with the standard database, the secondary spectrum of No. 10 unknown (retention time 86.49 minutes) is consistent with the standard secondary spectrum of bergamot. The map link and standard map of bergamot in the standard database are shown in FIG. 13. Therefore, it is speculated that the compound is bergamot, and its structural formula is shown in FIG. 14.

The information of bergamot compounds is as follows:
Chinese name: bergamot
English name: Bergamottin
Molecular formula: C21H2204
Molecular weight: 338.40
CAS number: 7380-40-7.

(5) Through the qualitative analysis of the above high-resolution mass

spectrometry and the comparison with the common characteristic peaks in the characteristic spectra of Fructus Cnidii, Fructus Cnidii total coumarin and Fructus

Cnidii total coumarin ointment, it was confirmed that the No. 10 peak, No.9 peak and No.9 peak in Fructus Cnidii total coumarin ointment were bergamot. This is the first time that bergamot has been identified in Fructus Cnidii.

### Example 4

Fructus Cnidii was first recorded in Shennong's Classic of Materia Medica, and listed as the top grade. It is bitter and pungent, warm in nature, and enters the spleen and kidney meridians. Functions: Warming kidney, strengthening yang, eliminating dampness, expelling wind and killing insects. Clinically, it is often used for impotence, infertility due to cold uterus, leukorrhagia due to cold and dampness, and lumbago due to dampness. External treatment of vulvar eczema, skin itching, women's vaginal itching, trichomonal vaginitis and so on. According to modern pharmacological research, Fructus Cnidii has antihistamine, antifungal and antitumor effects.

The therapeutic effects of the total coumarin ointment of Fructus Cnidii in Example 2 of this application on herpes zoster, psoriasis and atopic dermatitis are illustrated by clinical trials:

Patient 1, a 64-year-old male, complained of local skin pain (flattening), itching and burning sensation on the inner side of the left thigh (L3 single skin area) for 5 days, and rash and blisters for 2 days on Jan. 26, 2021.

Physical examination: A single skin area of the left L3 is red or dark red with blisters, and some blisters are clustered, umbilicus is depressed and scabs are formed. Local skin is squashed, two lymph nodes in the left groin are swollen, and the surface is smooth without adhesion, with mild tenderness.

### Clinical Diagnosis: Herpes Zoster

Treatment: Pikang cream was used for 2 days, followed by Piyanping for 3 days, and the symptoms did not relieve or even worsen. After topical treatment with total coumarin ointment of Fructus Cnidii twice a day for 3 days, the symptoms were obviously relieved. Continue medication: once day for 4 days; every other day for 8 days.

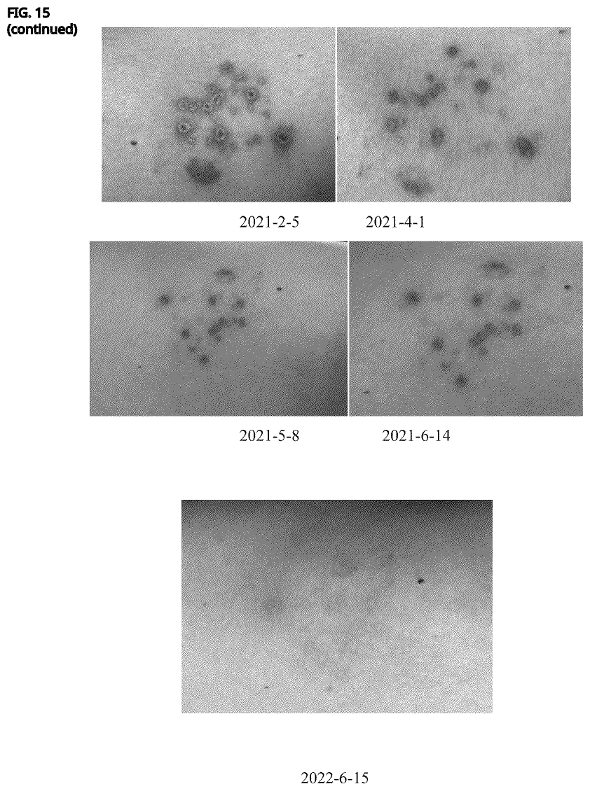

No recurrence has been found since the recovery (see FIG. 15 for details). There were no adverse reactions during the treatment.

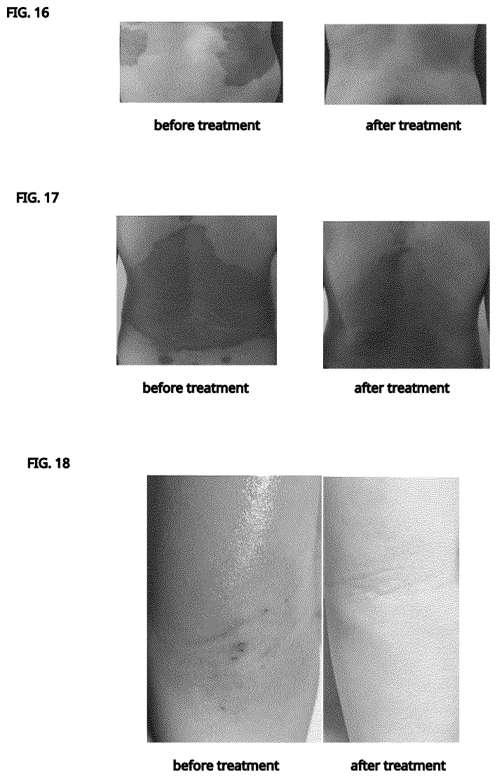

Patient 2, a 54-year-old male, suffered from psoriasis for 23 years, and various treatments had little effect. There is no recurrence after external treatment with total coumarin ointment of Fructus Cnidii (see FIG. 16-17 for details, in which FIG. 16 shows the abdomen and FIG. 17 shows the back). There were no adverse reactions during the treatment.

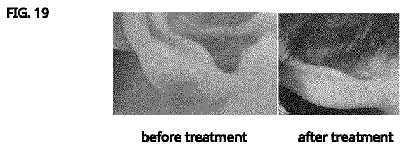

Patient 3, female, 5 years old, suffered from atopic dermatitis for 3 years. There is no recurrence after external treatment with total coumarin ointment of Fructus Cnidii (see FIG. 18-19 for details, in which FIG. 18 shows the knee fossa and FIG. 19 shows the ear). There were no adverse reactions during the treatment.

In addition, the Fructus Cnidii total coumarin containing bergamot and the Fructus Cnidii total coumarin preparation in this application have significant inhibitory effects on mitosis of mouse vaginal epithelial cells; It can obviously increase the number of scales formed by granular layer in rat tail skin scales; It has a strong inhibitory effect on passive skin allergic reaction in rats; It can obviously inhibit the ear swelling of mice caused by croton oil or the foot swelling of rats caused by egg white; It can significantly improve the itching threshold of guinea pigs caused by histamine phosphate, indicating that it has obvious itching relieving effect (the effect can be seen in the inventor's early application patents 20,041,0079250.5 and 02114903.8), further indicating that the Fructus Cnidii total coumarin containing bergamot and the Fructus Cnidii total coumarin preparation in this application have the effect of common Fructus Cnidii total extract. For allergic dermatosis (including eczema, atopic dermatitis, etc.), psoriasis and herpes zoster, it can change the pathological changes of these diseases, improve clinical symptoms, take effect quickly, have high curative effect and few adverse reactions, and at the same time, it has a local adjustment effect on human body.

To sum up, it is only a specific embodiment of the present invention, but the protection scope of the present invention is not limited to this. Any modification, equivalent substitution and improvement made by any person familiar with the technical field within the technical scope disclosed by the present invention are included in the protection scope of the present invention.

1. A total extract of Fructus Cnidii, which contains bergamot.

2. The total extract of Fructus Cnidii as claimed in claim 1, characterized in that it further contains Zanthoxylol, Xanthotoxin, Isoanisidin, Bergamot lactone, Imperatorin, Osthole, Hesperidine hydrate, and Hesperidone.

3. The application of the total extract of Fructus Cnidii as claimed in claim 1 or 2 in the preparation of drugs for the prevention and/or treatment of allergic dermatosis, psoriasis, or herpes zoster.

4. The application according to claim 3, characterized in that the allergic dermatosis comprises allergic contact dermatitis, atopic dermatitis, eczema, urticaria, and drug rash.

5. The application according to claim 3, characterized in that the drug comprises a total extract of Fructus Cnidii combined with a pharmaceutically acceptable carrier or excipient.

6. The application according to claim 3, which is characterized in that the drug is a fructus cnidii ointment, spray or liquid preparation containing the total extract of Fructus Cnidii.

7. A method for constructing a fingerprint of the total extract of Fructus Cnidii, characterized by comprising the following steps:
(1) Preparation of mixed reference solution: Take control samples of p-coumaric acid, osthol, isoanisidin, zanthoxylol, zanthoxyl, bergamot lactone, and imperatorin, dissolve them in ethanol to obtain a mixed reference solution;
(2) Preparation of test solution: Take the total extract of Fructus Cnidii, add ethanol, sonicate, supplement the lost mass with ethanol, mix well, filter, take the filtrate, and obtain the test solution;
(3) Take a mixed reference solution and a test solution, inject the sample, use liquid chromatography-high-resolution mass spectrometry full scan analysis, use software to identify common peaks in the characteristic spectrum of the test sample, extract and confirm the characteristic components, analyze, assign and confirm the unique chemical components of Fructus Cnidii, obtain the chemical components of Fructus Cnidii, and establish a standard fingerprint of the total extract of Fructus Cnidii.

8. A method for constructing a fingerprint of the total extract of Fructus Cnidii according to claim 7, characterized in that: in step (2), the volume percentage content of ethanol is 70%, the dosage relationship between the total extract of Fructus Cnidii and ethanol is 0.05 g: 50 mL, and the ultrasound treatment time is 60 minutes.

9. A method for constructing a fingerprint of the total extract of Fructus Cnidii according to claim 7, characterized in that: the parameters of liquid chromatography in step (3) are as follows: chromatographic conditions: C18 chromatographic column; Flow rate: 0.5 mL/min; Column temperature: 40° C.;
Wavelength: 310 nm; Injection volume: 10 μL; Mobile phase A: 0.1% (volume percentage content) acetic acid aqueous solution; Mobile phase B: methanol, gradient elution conditions are as follows:

| Time | Mobile phase | Mobile phase B |
|---|---|---|
| 0 | 95 | 5 |
| 10 | 65 | 35 |
| 20 | 55 | 45 |
| 35 | 55 | 45 |
| 40 | 53 | 47 |
| 65 | 53 | 47 |
| 70 | 30 | 70 |
| 75 | 10 | 90 |
| 85 | 10 | 90 |
| 85.1 | 0 | 100 |
| 120 | 0 | 100 |
| 120.1 | 95 | 5 |
| 130 | 95 | 5. |

10. A method for constructing a fingerprint of the total extract of Fructus Cnidii according to claim 7, characterized in that: in step (3), the standard fingerprint of Fructus Cnidii extract includes the following compounds: Zanthoxylol, Xanthotoxin, Isoanisidin, Bergamot lactone, Imperatorin, Osthole, Hesperidine hydrate, Hesperidone and Bergamot.
